SEQUENCE LISTING (7 sequences)

SEQ ID NO: 1 - protein, 38 aa, Artificial
  Example Sequence for Alignment Method
MISLIAALAVDVIMGRHTWESIVYEQFLPKAQHDLYIA

SEQ ID NO: 2 - protein, 39 aa, Artificial
  Example Sequence for Alignment Method
RSMLSIVAVCQNDAVIMGKKTWFSIVYAKAQHEKFVSPA

SEQ ID NO: 3 - protein, 36 aa, Artificial
  Example Sequence for Alignment Method
MISLIAALAVDARVIGMENAMPWNPADLAFKRNTLD

SEQ ID NO: 4 - protein, 36 aa, Artificial
  Example Sequence for Alignment Method
VKMISLIAALAVDRVIGMENAMPWNLPAFKAERNTL

SEQ ID NO: 5 - protein, 36 aa, Artificial
  Example Sequence for Alignment Method
AMISLIAALAVDRVIGMENAMPWNLPAWFKRNTLDV

SEQ ID NO: 6 - protein, 40 aa, Artificial
  Example Sequence for Alignment Method
SEAMISLIAALAVDRVIGMENAMPWNLPADLAWFKRNTLD

SEQ ID NO: 7 - protein, 42 aa, Artificial
  Example Sequence for Alignment Method
ASVVSLAAVCRNNKPEAVLMMKKSWFSLLYAKAQHEKFVSPV

(19) United States
(12) Patent Application Publication
(10) Pub. No.: US 2008/0177479 A1
(43) Pub. Date: Jul. 24, 2008

(54) DESCRIBING AND STORING METHOD OF ALIGNMENT INFORMATION

(75) Inventors: Tetsuro TOYODA, Tokyo (JP); Akiko Itai, Tokyo (JP)

Correspondence Address: GREENBLUM & BERNSTEIN, P.L.C., 1950 ROLAND CLARKE PLACE, RESTON, VA, 20191, US

(73) Assignee: INSTITUTE OF MEDICINAL MOLECULAR DESIGN, INC., Tokyo (JP)

(21) Appl. No.: 11/949,538

(22) Filed: Dec. 3, 2007

Related U.S. Application Data
(63) Continuation of application No. 10/985,984, filed on Nov. 12, 2004, now abandoned.
Application No. 10/985,984 is a continuation of application No. 09/869,312, filed on Nov. 8, 2001, now abandoned.

(30) Foreign Application Priority Data
Jan. 25, 1999 (JP) 11/15189

(51) Int. Cl. G01N 33/68; G06F 17/30

(52) U.S. Cl. 702/19; 707/104.1; 707/E17.002

(57) ABSTRACT

A description and storing method of an alignment information characterized by the separation of an alignment information on an amino-acid sequence or a nucleic-acid sequence into a sequence information and a gap information expressing correspondence between sequences.

### CROSS-REFERENCE TO RELATED APPLICATIONS

This application is a Continuation of application Ser. No. 10/985,984, filed Nov. 12, 2004, which is a Continuation of application Ser. No. 09/869,312, filed Jan. 25, 2000, which is a National Stage Application of International Application No. PCT/JP00/00355, filed Jan. 25, 2000, entering the National Stage on Nov. 8, 2001, and which claims priority of Japanese Application No. 11-15189, filed Jan. 25, 1999. The entire disclosures of U.S. patent application Ser. Nos. 10/985,984 and 09/869,312, and International Application No. PCT/JP00/00355 are considered as being part of this application, and both of the entire disclosures are expressly incorporated by reference herein in their entireties.

### FIELD OF INVENTION

The invention relates to describing and storing method of an alignment information with less data than its standard representation, where an alignment information is arranged in such a way as to correspond as many as possible in similar amino-acid residues among multiple amino-acid sequences or nucleic-acid residues among multiple nucleic-acid sequences.

### BACKGROUND ART

Information on sequences of nucleic acids bearing genetic information is translated to amino-acid sequences. Although functions and structure of a protein are determined by the sequence of 20 kinds of amino-acid residues, it is difficult to predict its functions and structure only from its amino-acid sequence. Thus, it requires a great deal of experimental effort for obtaining those kinds of knowledge. Recently as a result of genome analysis, many amino-acid sequences in various organisms have been determined without isolating the proteins experimentally. In spite of increased amount of sequence information, the functional roles of about half of the amino-acid sequences are still unknown. Therefore, informational techniques for studying the functions and structures of amino-acid sequences become more important.

Generally, it is possible to predict the functions and structure of a protein based on the empirical rule that the higher the similarity between the amino-acid sequences of two proteins, the higher the probability of having the same functions and similar structures. Moreover, it is also possible to estimate dendrograms of species depending on the similarities of the proteins extracted from those organisms. In those cases, it is important to create an “alignment” in which similar amino-acid residues among multiple sequences are corresponded as many as possible. The term “alignment” means processes of finding the correspondence of similar amino acids among the multiple sequences and also means a chart of the aligned sequences representing the correspondence as a result. In most cases, the correspondence of residues is evaluated by using score functions that reflect the similarity or distance of the residues. (The following explains the alignment of the amino acids in more details, where the term “alignment” used herein includes the alignment of the nucleic acids). Alignment information is expressed as aligned letters to represent the correspondence of amino-acid residues which are lined vertically (as a review, see, ┌3. Compare and Arrange Letters by Shigeki Mitaku and Minoru Kanehisa, Baifukan, 1995┘).

The alignment is a means to be utilized in science and industry, and studies which require alignment are increasing. Alignments are frequently carried out among the known proteins, for example, examining the relationship between functions and structures of multiple proteins with the same functions in different species, constructing a structure model of a protein based on crystal structures of similar proteins with known structures instead of crystal analysis. As a result, alignment results are often published on the journals of biochemistry, molecular biology, gene engineering and the like. However, the fact is that each researcher makes an alignment from the sequences as the need arises, and alignment information is often discarded after use. By making a database of alignment information, it is expected to apply the information to other study. Consequently, effectiveness of research will be promoted. Since the alignment technique has been almost standardized, it seems useful to add the alignment information among known proteins and newly known proteins by the genome analysis to the above database.

Although the standard representation of alignment information that expresses the gaps by inserting hyphens into the amino-acid sequence is convenient for a researcher to understand the similarity visually, the data structure of the representation is not appropriate for storing enormous alignment information compactly in a storage device, because it is necessary to record all letters expressing the residues of sequences and gaps. The total data size is at least (the number of residues in each sequence+the number of gaps)×number of sequences. The stored data of above form tends to be wasted from the point of information management, and also it leads to storage of redundant information because sequence information itself is usually obtained from the sequence information database. Since alignment information contains information of sequences therein, the redundancy of sequence information becomes terrible in many alignments among which the difference is only the locations of gaps in the same sequences.

In the future, the amount of amino-acid sequence information will increase at an accelerating speed and the use of alignment information will be more frequent. Consequently, it is required to develop a method which enables storage and search of an alignment information in storage devices. Furthermore, since nowadays computers are connected by network and data of alignment information are frequently transmitted and received by computers, more effective communication method of alignment information is necessary.

### DISCLOSURE OF THE INVENTION

An object of the present invention is to provide means of describing, storing and/or communicating alignment information effectively. More specifically, the object is to provide a method of describing, storing and/or communicating alignment information with small amount of data, which enables efficient search and editing of an alignment, and ready reproduction of a standard representation of alignment information as need arises.

Another object of the present invention is to provide a media or databases containing the alignment information and a media containing programs for implementation of the above means.

An alignment information is usually expressed by the correspondence in vertical direction of aligned amino-acid sequences, where amino acid residues with one-letter notation are placed horizontally in the order of “residue number” which is the order of the residue from the N-terminal in each sequence. Thus, the corresponding amino-acid residues are placed in the same column and each row includes residues of a sequence (Table 1). Hereinafter in the specification, this representation of the alignment information is called a “standard representation.” Amino-acid residues placed in the same column (vertical direction) are related to each other, and a hyphen is inserted when no corresponding residue exists in either sequence. Such a hyphen or a train of hyphens is called “gap region” or simply “gap.” “Number of residues in gap” is equal to the length of the gap or the number of the hyphens in the gap. Other regions except gaps are all corresponding.

TABLE 1

Standard representation of Alignment information
of amino-acid sequences (in which Sequence A is
SEQ ID NO: 1 and Sequence B is SEQ ID NO: 2)

Sequence A
--MISLIAALAVD-VIMGRHTWESIVYEQFLPKAQHDLYIA-
Sequence B
RSMLSIVAVCQNDAVIMGKKTWFSIVY----AKAQHEKFVSPA

The Inventors recognized that the alignment information can be separated into a “sequence information” and a “gap information” which contains information on the residue number at which each gap is inserted and the length of the residues of the gap, or the residue number and the number of residues of other sequences corresponding to the gap region of other sequence, or “correspondence information” which contains information on the residue number and the number of residues of each corresponding region. The sequence information is an array of characters in which each character represents one of 20 kinds of amino acids or 4 kinds of nucleic acids. The sequence information includes only the information on the sequence itself and does not include any information on correspondence with other sequences. The gap information or the correspondence information is a numerical data expressed by the residue number or the number of residues, both of which are equivalent information and mutually convertible. Consequently, it was found that a conversion of those separated forms of information to standard representation of alignment information was easily achieved by combining the gap information (or the correspondence information) with the sequence information.

Generally the number of gap regions in an alignment is small comparing with the number of amino-acid residues, which is about less than one-tenth. Therefore, it is possible to transmit on communication or to store alignment information effectively with very small amount of data by separating the gap information (or the correspondence information) and the sequence information from the alignment information and by storing or transmitting only the gap information (or the correspondence information). Furthermore, as it is often possible to obtain sequence information from available sequence database, only a gap information (or a correspondence information) can be handled to store or communicate an alignment information. When the sequence information is not readily available, the gap information together with the sequence information can be stored or transmitted. Since a sequence information is included in a standard representation of an alignment information, if this form of alignment information and sequence information is stored or transmitted, the sequence information will be treated redundantly. By separating a gap information and a sequence information, this sort of overlap will be avoided and effectiveness of storing or transmittance will be expected. The present invention was completed based on these findings.

The present invention thus provides a method of describing, storing, and/or transmitting an alignment information by separating into a sequence information and a gap information expressing correspondence between sequences, and a method of storing or transmitting an alignment information by separating into a sequence information and a correspondence information expressing correspondence between sequences. When the sequence information is available from existing databases, it is possible to separate an alignment information into a sequence information and a gap information or a correspondence information, and to store or transmit only the gap information or the correspondence information. The above-mentioned gap information or correspondence information does not include a sequence information, and can be expressed by a small amount of numerical data and converted to a standard representation of alignment information by computational calculation using the sequence information.

According to the present invention, the following methods are provided.

(1) A description method of an alignment information characterized by the separation of an alignment information on an amino-acid sequence or a nucleic-acid sequence into a sequence information and a gap information expressing correspondence between sequences;

(2) A storing method of alignment information characterized by the separation of an alignment information on an amino-acid sequence or a nucleic-acid sequence into a sequence information and a gap information expressing correspondence between sequences, wherein each information is stored and/or searched in one or more storage devices;

(3) The aforementioned method (2) which stores at least the gap information in one or more storage device;

(4) The aforementioned method (2) which stores only the gap information;

(5) Any one of the aforementioned methods (1) through (4) in which the gap information is described by using the residue number and/or the number of residues which indicates the location and the length of a gap region existing in the alignment information of more than two sequences, or numerical data convertible to those numbers by calculation;

(6) Any one of the aforementioned methods (1) through (4), in which the gap information is described by using a data containing the residue number of another sequence not included in the alignment or a virtual sequence or the column number of a standard representation of the alignment information or a numerical data convertible by calculation to those data;

(7) A method of generating the gap information of a new alignment information by calculation on only the gap information generated from an information of one or more alignments by any one of the aforementioned methods (1) through (6);

(8) A method of obtaining a standard representation of alignment information from both the gap information and the sequence information generated by any one of the aforementioned methods (1) through (7);

(9) A communication method of an alignment information characterized by the separation of an alignment information on an amino-acid sequence or a nucleic-acid sequence to a sequence information and a gap information expressing correspondence between sequences, and by the communication of the gap information at least out of these information;

(10) The aforementioned method (9) in which redundancy of sequence information is removed prior to communication and necessary minimum of the sequence information is transferred;

(11) A method of determining a substantially unique identifier of alignment information, wherein the identifier is determined using and depending on only and all of the data on the gap information and identifiers of sequences in the sequence information;

(12) A data record including at least its identifier and the gap information and the identifiers of sequences in the sequence information generated by any one of the aforementioned methods (1) through (10), and a media containing one or more of the record;

(13) The aforementioned data record (12), wherein its identifier is generated by the aforementioned method (11);

(14) A method of storing, searching, and/or communicating an alignment information which employs at least the aforementioned data record (12) or (13);

(15) Any one of the aforementioned methods (1) through (14) which employs the correspondence information instead of the gap information; and

(16) Any of the aforementioned methods (1) through (15) which employs “eigen-identifier” of a sequences in the sequence information.

The term “substantially unique identifier” in the above (12) is an identifier created in the same manner as the generation of “eigen-identifier” in the following explanation, except that the function takes a combined representation of both the gap information and identifiers of sequences in the sequence information as its input argument and standardization process is omitted.

From another aspect of the present invention, there are provided a storage method of an alignment information wherein the gap information or the correspondence information is stored in a database or a media; the aforementioned method wherein the gap information or the correspondence information together with the sequence information are stored in the database or the media; and aforementioned method wherein the gap information or the correspondence information is stored in the database or a distributed database or media.

From further aspect of the present invention, a method of communicating the necessary minimum of an information to reproduce an alignment information is provided. Specifically, there are provided a communication method of an alignment information wherein a gap information or a correspondence information is communicated; the aforementioned method wherein the sequence information together with the gap information or the correspondence information are communicated; and the aforementioned method wherein the sequence information together with the gap information or the correspondence information are communicated in the same mode or a diverged mode.

In addition to these inventions, there are provided a database of an alignment information including the gap information or the correspondence information; a database of an alignment information including the sequence information together with the gap information or the correspondence information; a database containing a separated alignment information into the sequence information and the gap information or the correspondence information; a media storing the gap information or the correspondence information; a media storing the sequence information together with the gap information or the correspondence information; and a media including separated alignment information into the sequence information and the gap information or the correspondence information. The kinds of the media are not particularly limited, and any kinds of medias can be used as storage devices such as memories, optic discs, magnetic discs, magnetic tapes, storage devices accessible by computers and the like, which are readily available to one of ordinary skill in the art. Furthermore, according to the present invention, a method of communicating the separated alignment information with the sequence information and the gap information or the correspondence information is provided.

### BEST MODE FOR CARRYING OUT THE INVENTION

The method of the present invention will be explained more specifically as to the application of the method to the alignment information obtained from two amino-acid sequences. However, the scope of present invention is not limited to the following embodiments or to any details of descriptions. Moreover, although the following explanations refers only to embodiments of separating alignment information into the sequence information and the gap information, it should not be interpreted that the method of the present invention is limited to those using the gap information, because the “gap information” and the “correspondence information” are equivalent and convertible mutually.

In general, the term “data record” used herein means a unit of storage including one or more representations of an information, an object of search (the data record being a file, a record in a database, or the like). The data records are usually stored in a media accessible by computers. The term “database” used herein means an apparatus comprising a storage means of one or more data records and an access means to any of the data records stored in media or storage devices.

The term “eigen-identifier of a sequence” means a substantially unique identifier of a sequence in a system wherein identifiers of the same sequences are the same and those of different sequences are substantially different (Japanese Patent Application No. (Hei)11-227438/1999). More specifically, the aforementioned eigen-identifier is computed by using a function whose domain is a set of various representations of the sequences and range is a set of their identifiers. For example, the function takes sequential characters expressing each residue in the sequence as its input argument. Then it converts them to a standard representation of the sequence (standardization process), because the same sequence can be represented in different notations. And the function transforms the representation to a sequence of bits of fixed length using one or more collision intractable hash functions, and then transforms the sequence of bits to a sequence of characters by converting each 4 or 5 bits to a single character. A preferred example of the hash functions includes SHA1, which has properties that takes arbitrary length of input data and gives 160-bit length of output value, and that SHA1 is virtually guaranteed to produce a different value from a different input data. Thus, regardless of the notation style of the representation of sequence information, the generated eigen-identifier becomes unique to the sequence information.

The term “sequence” used herein include both an amino-acid sequence and a nucleic-acid sequence unless otherwise specifically mentioned. The term “alignment information” used herein should be understood in the broadest sense including alignment results published on the journals and alignment results described by standard representations obtained from the standard methods, alignment results described by other expression other than the standard representation, alignment results produced as intermediate data in processes of various analyses, and partial informations of alignment results. The term “store” used herein includes the use of stored data records, as identity means of the data records in databases, existence means for determining whether or not the data record exists in databases, associating means among the data records by using their identifiers, search means for the data records in databases, copying means of any one of the data records to other databases, duplication means of all of the data records in databases, backup means of the data records by duplication means, access means of the data records in databases, recovery means from storage created by backup means, integration means of more than two data records to a single data record and the like. The term “communication” used herein means copying of a data from a location (sender) to another location (receiver) in a system, where the data is stored in a media or a storage device at each location. Thus, a sender and a receiver may be different memory addresses of the same computer, or may be different computers joined by networks.

By removing the sequence information from the alignment information on the two sequences A and B as shown in Table 1, the information shown in Table 2 is obtained. The number indicates the residue number, and the kind of amino acid corresponding to each residue number can be deducted from the amino-acid sequence information. Therefore, in the methods of present invention, the location and the length of the gap region expressed by hyphens shown on Table 2 are described and stored in other forms.

TABLE 2

| | |
|---|---|
| A | — — 1 2 3 4 5 6 7 8 9 10 11 — 12 13 14 15 16 17 18 |
| B | 1 2 3 4 5 6 7 8 9 10 11 12 13 14 15 16 17 18 19 20 21 |
| A | 19 20 21 22 23 24 25 26 27 28 29 30 31 32 33 34 35 36 37 — |
| B | 22 23 24 25 26 27 — — — — 28 29 30 31 32 33 34 35 36 37 |

The method of the present invention is characterized by storing an alignment information using a small amount of numerical data without the sequence information shown in Table 2, and as a means for that, it employs a “gap information” to represent correspondences between the sequences. In the following, a practical method of extracting a gap information from the alignment information is explained. However, the kinds or the representation of a gap information are not limited to the following method. As a gap information, any information which represents correspondence between sequences may be utilized. Information on the number of residues on each sequence is assumed to be included in the sequence information in the following explanation.

### Representation of Gap Information

Alignment indicates relative relationship between multiple sequences. The location and the length of a gap region in an aligned sequence of an alignment can be represented by the residue number indicating the location of the region and the number of hyphens contained in the region. Representation methods are not limited, therefore any method may be employed. Some typical embodiments are given in the following, however, the representation methods are not limited to these embodiments. Methods are divided roughly into following \textcircled{1}, \textcircled{2}, and \textcircled{3}. Method \textcircled{1} is a representation based on the residue number of one selected sequence among multiple sequences included in the alignment. Usually, the first sequence in the alignment is selected. Method \textcircled{2} is represented by using the column number of the alignment described by the standard representation, or by using the residue number of a virtual sequence which is not a real sequence included in the alignment but has no gap and the same length as column length of the alignment, thus its residue number serves as the same as the column number of the alignment. In method \textcircled{3}, gap information is represented by placing the number of residues of the gap region and the residue region alternately for each sequence of the alignment. In each method, various modifications and alternations are possible. In case when the gap information is expressed by the number of residues only, it is desirable to describe gap regions in the increasing order of residue number.

Assume sequence A as the selected sequence, gap information of the alignment on Table 2 is represented as ┌2, 11, 1, 13, −4, 9, 1┘ by arranging each residue number alternately (residue number of gap region first in this example) of the gap region and the corresponding region (method \textcircled{1}a). In order to show where the gap region exists in which sequence, the residue number of the gap in sequence B is expressed as a negative number by putting a minus sign. This representation means that from the left end gap of 2 residues in sequence A, corresponding region of 11 residues, gap of 1 residue in sequence A, corresponding region of 13 residues, gap of 4 residues in sequence B, corresponding region of 9 residues, and gap of 1 residue in sequence A at the end are lined up.

Alignment shown in Table 2 may also be represented as ┌0, 2, 11, 1, 24, −4, 37, 1┘ by using residue number and the number of residues when the residue number is placed first (method \textcircled{1} b). This representation expresses each gap region by the residue number just before it, and the number of residues included in the gap region. The gap in sequence B is distinguished from the sequence A by the negative number. It means that gap of 2 residues in residue number 0 (N-terminal) of sequence A, gap of 1 residue after the number 11 of sequence A, gap of 4 residues in sequence B after the corresponding location to the residue number 24 of sequence A, gap of 1 residue after the number 37 of sequence A are lined.

As for the representation by method \textcircled{1}a and method \textcircled{1}b, by addition of the number of residues of the corresponding region to the residue number in method \textcircled{1}a, the representation will be converted to that obtained by method \textcircled{1}b. For example, ┌2, 11, 1, 13, −4, 9, 1┘ in method \textcircled{1}a is converted to representation of method \textcircled{1}b by placing 0 to the first gap (because the first gap starts with 0) and leaving 11 as it is, and changing 13 to 24 (=11+13), 9 to 37 (=24+4+9) (because 4 is a gap of sequence B and residues exist in sequence A). Thus, the representation is converted to ┌0, 2, 11, 1, 24, −4, 37, 1┘. By carrying out the procedure in reverse, conversion of \textcircled{1}b to \textcircled{1}a is possible.

However, in the case of representing alignment information including 3 or more sequences as shown in Table 3 or adding or in the case of deleting sequences in alignment, it is convenient to treat all sequences equally for reproducing standard representation. In the following, using examples (Table 3) of alignment containing 3 sequences, methods \textcircled{2} and \textcircled{3} will be explained. These methods treat all sequences equally when converting to standard representation, deleting sequences and integrating alignments.

TABLE 3

| | |
|---|---|
| A | — — 1 2 3 4 5 6 7 8 9 10 11 — 12 13 14 15 16 17 18 19 20 |
| B | 1 2 3 4 5 6 7 8 9 10 11 12 13 14 15 16 17 18 19 20 21 22 23 |
| C | — 1 2 3 4 5 6 7 8 9 10 11 12 13 14 15 16 17 18 19 20 21 22 |
| A | 21 22 23 24 25 26 27 28 29 30 31 32 33 34 35 36 37 — |
| B | 24 25 26 27 — — — — 28 29 30 31 32 33 34 35 36 37 |
| C | 23 24 25 — — — 26 27 28 29 30 31 32 33 34 35 36 — |

The number of columns in a standard representation of an alignment information is usually larger than the number of the residues of the longest sequence due to gap. For the representation of the alignment in Table 3, a serial number is given to the columns as shown in Table 4 (rows of column numbers are indicated as R)

TABLE 4

| | |
|---|---|
| R | 1 2 3 4 5 6 7 8 9 10 11 12 13 14 15 16 17 18 19 20 21 22 |
| A | — — 1 2 3 4 5 6 7 8 9 10 11 — 12 13 14 15 16 17 18 19 |
| B | 1 2 3 4 5 6 7 8 9 10 11 12 13 14 15 16 17 18 19 20 21 22 |
| C | — 1 2 3 4 5 6 7 8 9 10 11 12 13 14 15 16 17 18 19 20 21 |
| R | 23 24 25 26 27 28 29 30 31 32 33 34 35 36 37 38 39 40 41 |
| A | 20 21 22 23 24 25 26 27 28 29 30 31 32 33 34 35 36 37 — |
| B | 23 24 25 26 27 — — — — 28 29 30 31 32 33 34 35 36 37 |
| C | 22 23 24 25 — — — 26 27 28 29 30 31 32 33 34 35 36 — |

Method \textcircled{2}

Based upon the column numbers (R as in Table 4), the location of residues or gaps in each sequence are expressed by method \textcircled{2}. When the first and the last column numbers of the residues from the N-terminal are described by this method, the alignment in table 3 is represented as follows:

| | |
|---|---|
| Sequence A | 3, 13, 15, 40 |
| Sequence B | 1, 27, 32, 41 |
| Sequence C | 2, 26, 30, 40 |

In sequence A, residues exist between the column numbers 3 and 13, 15 and 40. For sequence B, residues exist between the column numbers 1 and 27, 32 and 41. For sequence C, residues exist between the column numbers 2 and 26, 30 and 40.
Method \textcircled{3}

For each sequence, it is possible to represent the gap information by indicating the number of residues of each gap and corresponding residues alternately without using any specifically selected sequence or column number as basis (method \textcircled{3}). According to this method, when the number of residues of gap region is shown first, Table 3 alignment can be described as follow:

| | |
|---|---|
| Sequence A | 2, 11, 1, 26, 1 |
| Sequence B | 0, 27, 4, 10 |
| Sequence C | 1, 25, 3, 11, 1 |

In the gap information of sequence A, 2, 1, and 1 show the number of residues in each gap, and continuous 11 and 26 amino-acid residues are placed between the gaps. In sequence B, after the 27 residues from the N-terminal, 10 residues exist across the gap of 4 residues. In sequence C, 25 residues exist after the gap of 1 residue of N-terminal, 11 residues exist after the gap of 3 residues, and gap of 1 residue at the end exist. It is also possible to begin with the number of corresponding residues first.

### Conversion to Standard Representation of Alignment Information

A gap information represented by any method can be converted to a standard representation of an alignment information. First, it is necessary to calculate the number of columns required to enumerate all residues including the gap and to prepare columns. Following the gap information, the alignment information in Table 3 will be obtained when the residue numbers or hyphens are corresponded in each column. Moreover, the standard representation of alignment information will be reproduced when amino-acid residues are placed into the corresponding residue number of each sequence.

The alignment information shown in Table 3 will be reproduced as follows. From the gap information in Table 2 according to alignment by method \textcircled{2} (sequence A ┌3, 13, 15, 40┘, sequence B ┌1, 27, 32, 41┘, sequence C ┌2, 26, 30, 40┘), necessary number of columns is determined as equal to the largest column number, i.e., 41. After 41 columns are prepared, for sequence A all 37 residues are placed in each column from the N-terminal in columns from numbers 3 to 13 and 15 to 40. For sequence B, all 37 residues are placed in each column from the N-terminal in columns from numbers 1 to 27 and 32 to 41, and for sequence C, all 36 residues are placed in each column from number 2 to 26 and 30 to 40.

From the gap information shown in Table 3 according to alignment by method \textcircled{3} (sequence A ┌2, 11, 1, 26, 1┘, sequence B ┌0, 27, 4, 10┘, sequence C ┌1, 25, 3, 11, 1┘, necessary number of columns is calculated as 2+11+1+26+1=41 by adding all residue numbers of sequence A. The same number is obtained by calculation based on sequence B (27+4+10=41) or sequence C (1+25+3+11+1=41). In these columns, for sequence A, 11 residues after the gap of 2 residues, 26 residues after the gap of 1 residue, and a gap of 1 residue are lined in order from the N-terminal. For sequence B, 27 residues are lined in order from the N-terminal, and 10 residues after the gap of 4 residues are lined. The same procedure is applies to sequence C.

Generally, the column length of an alignment often changes in a standard representation of an alignment by modification such as deletion of one or more of the sequences contained or addition of other sequences. The change of the column length of an alignment is caused dependently on the change of insertion mode of gaps, whilst independently on the sequence information. The calculation method of the present invention is characterized in that the gap information of a modified alignment is easily converted from a gap information of one or more original alignments, with no sequence information required.

Extraction of Sequence from Alignment Information

The following is a procedure of extracting alignment information of A and C according to the standard representation from the alignment information shown in Table 3 by removing one part of sequence, for example, sequence B. From the gap information set out in Table 3 according to alignment by method \textcircled{2} (Sequence A ┌3, 13, 15, 40┘, Sequence B ┌1, 27, 32, 41┘, and Sequence C ┌2, 26, 30, 40┘), the information on sequence A and sequence C ┌3, 13, 15, 40┘, ┌2, 26, 30, 40┘) is extracted. In the columns from 1 to 41, column numbers which correspond to the gap in both sequences (in this case 1 and 41) are searched operationally. In the example, the smallest number of the gap information is 2 in sequence C, therefore, decrement of all the numbers is made so that the smallest becomes 1 which is the starting end of alignment. Gap informations of sequence A and C are 2, 12, 14, 39┘ and ┌1, 25, 29, 39┘, respectively. The gap region between column number 12 and 14 of sequence A does not overlap with the gap region between column number 25 and 29 of sequence C. As a result, the number of columns becomes 39, and these column numbers are moved to the left end (small column number side). The alignment obtained from the calculated gap information is shown in Table 5.

TABLE 5

| | |
|---|---|
| R | 1 2 3 4 5 6 7 8 9 10 11 12 13 14 15 16 17 18 19 20 21 22 |
| A | — 1 2 3 4 5 6 7 8 9 10 11 — 12 13 14 15 16 17 18 19 20 |
| C | 1 2 3 4 5 6 7 8 9 10 11 12 13 14 15 16 17 18 19 20 21 22 |
| R | 23 24 25 26 27 28 29 30 31 32 33 34 35 36 37 38 39 |
| A | 21 22 23 24 25 26 27 28 29 30 31 32 33 34 35 36 37 |
| C | 23 24 25 — — — 26 27 28 29 30 31 32 33 34 35 36 |

As for the gap information for the alignment shown in Table 3 by method \textcircled{3} (sequence A ┌2, 11, 1, 26┘, sequence B ┌0, 27, 4, 10┘, sequence C ┌1, 25, 3, 11, 1┘, after the conversion to gap information by method \textcircled{2}, extraction of sequences will be easily conducted by the calculation of gap information as explained above.

For sequence A, an example of conversion from the gap information by method \textcircled{3} to that by method \textcircled{2} will be shown below. The gap information obtained by method \textcircled{3} ┌2, 11, 1, 26, 1┘ describes the number of residues of gap region s and residue regions alternately, and there are two places where the residues exist. The first and the last column numbers of each region can be calculated as 2+1=3, 2+11=13 and 2+11+1+1=15, 2+11+1+26=40, and accordingly, it is possible to convert the information to the gap information by method \textcircled{2}, i.e., ┌3, 13, 15, 40┘. When the same procedure is applied to sequence B, the gap information by method \textcircled{3} ┌0, 27, 4, 10┘ can be converted to the gap information by method \textcircled{2} ┌1, 27, 32, 41┘ by the operation of 0+1=1, 0+27=27, 0+27+4+1=32, 1+27+4+10=41.

### Integration of Multiple Alignment Information

According to the methods of present invention, more than two alignment results where common sequences exist, such as shown in Table 6, can be easily integrated by the calculation of gap information.

TABLE 6
Alignment 1

| | |
|---|---|
| A | — — 1 2 3 4 5 6 7 8 9 10 11 — 12 13 14 15 16 17 18 19 20 |
| B | 1 2 3 4 5 6 7 8 9 10 11 12 13 14 15 16 17 18 19 20 21 22 23 |
| C | — 1 2 3 4 5 6 7 8 9 10 11 12 13 14 15 16 17 18 19 20 21 22 |
| A | 21 22 23 24 25 26 27 28 29 30 31 32 33 34 35 36 37 — |
| B | 24 25 26 27 — — — — 28 29 30 31 32 33 34 35 36 37 |
| C | 23 24 25 — — — 26 27 28 29 30 31 32 33 34 35 36 — |

Alignment 2

| | |
|---|---|
| A | — — — 1 2 3 4 5 6 7 8 9 10 11 12 13 14 15 16 17 18 19 20 |
| D | 1 2 3 4 5 6 7 8 9 10 11 12 13 — 14 15 16 17 18 19 20 21 22 |
| A | 21 22 23 24 25 26 27 28 29 30 31 32 33 34 35 36 37 — |
| D | 23 24 25 — — 26 27 28 29 30 31 32 33 34 35 36 37 — |

The gap information in alignment 1 by method \textcircled{2} is ┌3, 13, 15, 40┘ for sequence A, ┌1, 27, 32, 41┘ for sequence B, and ┌2, 26, 30, 40┘ for sequence C, and the gap information in alignment 2 is ┌4, 40┘ for sequence A and ┌1, 13, 15, 26, 29, 40┘ for sequence D. From the common gap information of sequence A, it can be understood that placements of new gap of 1 residue to the N-terminal for alignment 1 and new gap of 1 residue between column numbers 14 and 15 for alignment 2 are required.

Therefore, in all sequences contained in alignment 1, as a result of adding 1 to each column number due to the N-terminal gap, the gap information for sequence A is ┌4, 14, 16, 41┘, for sequence B ┌2, 28, 33, 42┘, and for sequence C ┌3, 27, 31, 41┘. For alignment 2, due to the introduction of the new gap between the column numbers of 14 and 15, the gap information for sequence A is ┌4, 14, 16, 41┘ and for sequence D ┌1, 13, 16, 27, 30, 41┘. Thus, in both alignments, identical gap informations on sequence A are obtained. If the above information is converted to a standard representation according to the above mentioned procedures, integrated alignments shown in Table 7 are obtained. The required number of columns in the integrated alignments is 42 based on the largest column number of sequence B.

TABLE 7

| | |
|---|---|
| R | 1 2 3 4 5 6 7 8 9 10 11 12 13 14 15 16 17 18 19 20 21 22 |
| A | — — — 1 2 3 4 5 6 7 8 9 10 11 — 12 13 14 15 16 17 18 |
| B | — 1 2 3 4 5 6 7 8 9 10 11 12 13 14 15 16 17 18 19 20 21 |
| C | — — 1 2 3 4 5 6 7 8 9 10 11 12 13 14 15 16 17 18 19 20 |
| D | 1 2 3 4 5 6 7 8 9 10 11 12 13 — — 14 15 16 17 18 19 20 |
| R | 23 24 25 26 27 28 29 30 31 32 33 34 35 36 37 38 39 40 41 42 |
| A | 19 20 21 22 23 24 25 26 27 28 29 30 31 32 33 34 35 36 37 — |
| B | 22 23 24 25 26 27 — — — — 28 29 30 31 32 33 34 35 36 37 |
| C | 21 22 23 24 25 — — — 26 27 28 29 30 31 32 33 34 35 36 — |
| D | 21 22 23 24 25 — — 26 27 28 29 30 31 32 33 34 35 36 37 — |

For the gap information according to method \textcircled{3}, an integration by calculation is possible as explained above by converting to a gap information by method \textcircled{2}. When no common sequence exists, at least one sequence is chosen from each alignment and then an alignment of those sequences is created so that a new alignment shares the same sequences with all the alignments.

In the methods of the present invention, the numbers representing a gap information are preferred to be expressed in the unit of byte to be processed effectively by a computer. For instance, a number which can be expressed within 1 byte is expressed by 1 byte, and when it is not possible to express by 1 byte, it can be expressed by multiple bytes. To show how many bytes are used for expression, a flag may be put to a specific bit. Instead of + and − signs, sign bit may be put in the data. By simple algorithm, other data converted from a group of these numbers may also be utilized as gap information.

It is necessary to put symbols or numbers (identifiers of sequences) bound to the sequence upon separating a sequence information from an alignment and storing of the gap information in a database. It is possible to give symbols or numbers (identifiers of alignments) bound to respective alignment informations and search sequence identifiers form the alignment identifiers utilizing databases. It is also possible to store a gap information belonging to the same alignment in the same data record in various formats such that numbers used in a representation of each gap information are linked together to clarify the partition.

It is preferred that a data record includes an identifier of an alignment as the identifier of the data record. One of preferred embodiments of a gap information stored is a data record which includes at least its identifier and a certain representation of a gap information and identifiers of sequences contained in the alignment to which the gap information corresponds. By using the data records, a gap information corresponds one-to-one to an alignment information uniquely. Because a data record includes a gap information and identifiers corresponding to a sequence information, the same identifier of the alignment information can be used as the identifier of the data record. The identifier of the alignment information must be unique to the information specifying the alignment. A property required to the identifiers is that the identifier depends upon only and all of the data on the gap information and identifiers of sequences in the sequence information.

Sequence information is not necessarily stored in the files, tables or database containing gap information. Although it is preferable to store a sequence information in the same media as that stores a gap information, a sequence information may be stored in other medias available for searching. When the sequence information stored in other database is available, only the gap information may be stored in the database. It is also possible to store some sequence informations frequently used together with the gap information, and to utilize an outside database for other sequence informations. Furthermore, a sequence information in the database may include species and organisms and subtypes in addition to the sequence identifier, the name of proteins, the number of amino acid residues, amino-acid sequence, and the like. The information may be administered separately by the tables of a relational database.

### Communication Method

It is possible to communicate an alignment information effectively by transmitting the alignment information from a sender to a receiver in a separated form of a sequence information and a gap information. First, according to the methods described above, an alignment information is separated to a sequence information and a gap information. A sequence identifier of an amino-acid sequence corresponding to the gap information is added to the gap information, which is transmitted from the sender to the receiver. If an amino-acid sequence information corresponding to the sequence identifier in a receivers' database is available, the information at the receiver's end will be utilized. If that data is not available to the receiver, a request for sequence information corresponding to the sequence identifier is sent by the receiver to the sender, or alternatively, the receiver obtains sequence information corresponding to the sequence identifier from other available database. The receiver can reconstruct the alignment information from the gap information according to the methods explained above.

According to another method, an alignment information is first separated into a sequence information and a gap information. A sequence identifier which is able to be used to identify an amino-acid sequence corresponding to the gap information is added to the gap information, and then the resulting information is transmitted from the sender to the receiver. The redundancy of the sequences is reduced. Necessary minimum of the sequences for the gap information are transmitted to the receiver automatically or upon a request. So far as the gap information and sequence information are separated, the order of transmittance is not limited.

### EXAMPLES

The present invention will be explained more specifically with reference to examples. However, the scope of present invention is not limited to the following examples. In the following examples, gap information presented by method (3 was employed as a preferred mode of the present invention. It should be understood that alignment information may be separated to gap information and sequence information by other methods specifically explained above or those not disclosed in the specification.

### Example 1

An alignment information on 4 amino-acid sequences was divided to the gap information and the sequence information as shown in Table 8, and the gap information was stored in the database. In the Table, each amino-acid sequence was specified by giving the sequence identifier. The term “ID” herein means identifier. In the gap information, sequence ID=000001 represents the selected sequence as a basic sequence, and the gap information of sequence ID=000002 through 000004 are represented relative to the selected sequence.

TABLE 8
(Gap Information)

| | |
|---|---|
| Sequence ID | Gap Information |

| | |
|---|---|
| 000001 | 3, 11, 1, 26, 1 |
| 000002 | 1, 27, 4, 10, 0 |
| 000003 | 2, 25, 3, 11, 1 |
| 000004 | 0, 13, 2, 12, 2, 12, 1 |

(Sequence Information)

| Sequence ID; | Protein Name; | Number of amino acid residue; | and Amino-acid sequence |
|---|---|---|---|
| 000001 | xxxxxxxx | 37 | MISLIAALAVDARVIGMENAMPWNPADLAFKRNTLD (SEQ ID NO. 3) |
| 000002 | xxxxxxxx | 36 | VKMISLIAALAVDRVIGMENAMPWNLPAFKAERNTL (SEQ ID NO. 4) |
| 000003 | xxxxxxxx | 36 | AMISLIAALAVDRVIGMENAMPWNLPAWFKRNTLDV (SEQ ID NO. 5) |
| 000004 | xxxxxxxx | 37 | SEAMISLIAALAVDRVIGMENAMPWNLPADLAWFKRNTLD |
| | | | (SEQ ID NO. 6) |

### Example 2

The property information in the columns of alignment (X) in Table 9 is integrated with alignment (Y) in Table 10 and marked.

TABLE 9
Alignment information (X)

Column Property Information
--*-*--*----#-#***--#*-****-----****------
Sequence A
--MISLIAALAVD-VIMGRHTWESIVYEQFLPKAQHDLYIA-
(SEQ ID NO. 1)
Sequence B
RSMLSIVAVCQNDAVIMGKKTWFSIVY----AKAQHEKFVSPA
(SEQ ID NO. 2)

TABLE 10
Alignment information (Y)
Sequence B
-RSMLSIVAVCQN---DAVIMGKKTWFSIVYAKAQHEKFVSPA
(SEQ ID NO. 2)
Sequence C
A-SVVSLAAVCRNNKPEAVLMMKKSWFSLLYAKAQHEKFVSPV
(SEQ ID NO. 7)

In Table 9, the positions in which amino-acid sequences are the same in the correspondence of columns in sequence A and sequence B are indicated as *. Also the positions of amino acids which are functionally important are marked as #. According to the same procedure of separating the alignment information into the sequence information and the gap information as shown in Table 8, the column property information is separated to the property kind information and the column location information as shown in Table 11 by regarding the [-] as gap in the column property information on Table 9. In this case, the column location information is the same as the gap information expressed by method \textcircled{3}.

TABLE 11

| Property kind information | ***##***#********* |
|---|---|
| Column location information | 2, 1, 1, 1, 2, 1, 4, 1, 1, 4, 2, 2, 1, 4, 5, 4, 7 |

Table 12 shows the results of calculated column location information in alignment (Y) shown in Table 10 from the column location information shown in Table 11, the gap information on sequence B shown in Table 9, and the gap information on sequence B shown in Table 10.

TABLE 12

| | |
|---|---|
| Column location information | 3, 1, 1, 1, 2, 1, 7, 1, 1, 4, 2, 2, 1, 4, 1, 4, 7 |

Table 13 shows the column property information on alignment (Y) obtained from the column location information on Table 12 and the property kind information on Table 11. As it is clear from comparison of Table 9 with Table 13, the column property information is corresponded on sequence B which is common to alignment (X) and alignment (Y).

TABLE 13

Alignment information (Y) which is mapped so as to
correspond with the column information of
alignment information (Y) on sequence B.

Column property information
---*-*--*-------#-#***--#*-****-****-------
Sequence B
-RSMLSIVAVCQN---DAVIMGKKTWFSIVYAKAQHEKFVSPA
(SEQ ID No. 2)
Sequence C
A-SVVSLAAVCRNNKPEAVLMMKKSWFSLLYAKAQHEKFVSPV
(SEQ ID NO. 7)

### Example 3

It is demonstrated that representation of the alignment information shown in Table 14 is separated to the sequence information (Table 15) and the gap information. The gap information is stored as a data record (Table 16) containing its identifier, the gap information, identifiers of sequences, date and the like. The identifiers of sequences are eigen-identifiers, and the identifier of the data record is determined using and depending on only and all of the data on the gap information and identifiers of the sequences. The data in Table 14 to 16 is written by XML (extensible markup language).

TABLE 14

Sequence P
-RSMLSIVAVCQN---DAVIMGKKTWFSIVYAKAQHEKFVSPA
(SEQ ID NO. 2)
Sequence Q
A-SVVSLAAVCRNNKPEAVLMMKKSWFSLLYAKAQHEKFVSPV
(SEQ ID NO. 7)

In Table 15, sequence information of sequence P and Q is shown. Each sequence is tagged by “{sequence}” and “{/sequence}” at its head and tail. The start-tag at the head indicates the start of the sequence and the end-tag at the tail indicates the end of the sequence. In the start-tag {sequence}, “ed=” is an attribute that indicates the eigen-identifier of the sequence between the tags. Thus, the eigen-identifier of sequence P is:

“SA16rxgd7d4xxgmjcuaf8v3f6crqu8p9bck.”
The eigen-identifier of sequence Q is:
“SA1j1r9pr0f9xcc00p57xke0kdijp8jvrh4.”

TABLE 15

{sequence ed =
“SA16rxgd7d4xxgmjcuat8v3f6crqu8p9bck”}
RSMLSIVAVCQNDAVIMGKKTWFSIVYAKAQHEKFVSPA
(SEQ ID NO. 2)
{/sequence}
{sequence ed =
“SA1j1r9pr0f9xcc00p57xke0kdijp8jvrh4”}
ASVVSLAAVCRNNKPEAVLMMKKSWFSLLYAKAQHEKFVSPV
(SEQ ID NO. 7)
{/sequence}

TABLE 16

{record ed=“ AL17vr5emniqtmnj36eir4r0vbny5ym1nj9”
date=“19990507”}
{reference gap=“1,12,3,27” order=“1”}
SA16rxgd7d4xxgmjcuaf8v3f6crqu8p9bck
{/reference}
{reference gap=“0,1,1,41” order=“2”}
SA1j1r9pr0f9xcc00p57xke0kdijp8jvrh4
{/reference}
{/record}

In Table 16, the data record including gap information is shown. The data record starts from {record} tag, and ends at {/record} tag. Thus, the data record is consisted of whole letters in Table 16. In the data record, the identifier of each sequence is placed between {reference} and {/reference}. In each {reference} tag, gap information (method \textcircled{3}) is expressed by “gap=.” The “order=” in each {reference} tag indicates the dictionary order of the identifier in the data record.

The “ed=” in the {record} tag indicates the identifier of the record. The identifier is included in the data record as shown in Table 16. The following explains that the identifier is substantially unique identifier of the data record, where the date information does not effect on its uniqueness. In order to generate the identifier based on the gap information and identifiers of the sequences, each sequence identifier is joined with the letters in its “gap=,” and then they are joined in the order of dictionary. Thus after above procedures, it become one string of letters as shown in Table 17. The string is unique to the gap information and the identifiers of sequences, because the string is created from them. The string is then transformed by SHA-1 to 160-bit substantially unique data. Each 5 bits of the 160-bit data converted to one of “0, 1, 2, 3, 4, 5, 6, 7, 8, 9, a, b, c, d, e, f, g, h, i, j, k, x, m, n, y, p, q, r, s, t, u, v,” yielding 32-letter unique word. Finally, the substantially unique identifier is created by the word which is joined after “AL1” meaning the identifier is unique to the alignment information. Since the identifier is unique to the alignment information, it is favorable to use it as that of the data record.

TABLE 17

“SA16rxgd7d4xxgmjcuaf8v3f6crqu8p9bck1,12,3,27
SA1j1r9pr0f9xcc00p57xke0kdijp8jvrh40,1,1,41”

In this case, the alignment shown in Table 14 is separated to data including sequence information (Table 15) and data including gap information (Table 16). If each data is stored in different data records such as files, records of tables of databases and the like, it is apparent that the alignment is stored by one of the method of the present invention. Moreover, even if both of the data shown in Table 15 and Table 16 are stored in a single data record, it is considered that the alignment is also separately stored by the method, because sequence information and gap information is apparently separated to sections by the {sequence} . . . {/sequence} tags and {record} . . . {/record} tags.

### INDUSTRIAL APPLICABILITY

By the methods of present invention, an alignment information is stored with very small amount of data as a whole, because a sequence information is not overlapped and a gap information is expressed by several numbers when alignment information is stored. Furthermore, a standard representation of an alignment information can be reproduced easily from those information. Moreover, by calculating the correspondence information of one or more alignments, it is possible to edit the alignment and to create an integrated representation of alignment information, and also various applications other than the reuse of the alignment information are possible. Accordingly, according to the methods of the present invention, storing efficiency of an alignment information to database and various medias (for example, magnetic-recording media, optical-recording media, and the like) will make great strides, and it is possible to store a huge amount of alignment informations and prepare database more effectively for the reuse of those alignment informations.

Furthermore, since a sequence information and a gap information are administered separately, it will be easy to preserve the consistency and maintenance of database. Particularly in a relational database, possibility of the use of database will increase because the data is treated in more canonical conditions. Moreover, when the alignment information is transmitted according to the methods of present invention, receivers do not have to receive the sequence information already at hand, and accordingly, communication efficiency will improve and the overlap of the sequence information on the receiver side will be avoided. Particularly, it is effective when a huge amount of alignment informations is transmitted, duplication of database through communication is made, and the alignment information in the client-server system and communication between applications is exchanged.

1. A computer-implemented method of storing the necessary minimum of information to reproduce alignment information on amino acid sequences or nucleic acid sequences, comprising the steps of
a) generating information which represents location and length of a gap region or a corresponding region based on the alignment information; and
b) storing the information generated in step a) in at least one storage device.

2. A computer-implemented method of displaying multiple sets of alignment information on amino acid sequences or nucleic acid sequences, comprising the steps of:
a) obtaining sequence information on amino acid sequences or nucleic acid sequences constituting the alignment;
b) obtaining the information stored by the method according to claim 1;
c) calculating gap information or correspondence information of an integrated alignment for the multiple sets of alignment information based on the information obtained in step b); and
d) generating and displaying a standard representation of the integrated alignment information based on the sequence information obtained in step a) and the gap information or correspondence information calculated in step c).

3-5. (canceled)

6. The method according to claim 1, wherein the information generated in step (a) comprises data containing residue number of another sequence not included in the alignment or a virtual sequence or column number of a standard representation of the alignment information or a numerical data convertible by calculation to the data.

7-8. (canceled)

9. A method of communicating alignment information on amino acid sequences or nucleic acid sequences, comprising the steps of communicating the information generated or stored by the method according to claim 1.

10-16. (canceled)

17. A computer-implemented method of displaying alignment information on amino-acid sequences or nucleic acid sequences, comprising the steps of:
a) obtaining sequence information on amino-acid sequences or nucleic acid sequences constituting the alignment; and
b) generating and displaying a standard representation of the alignment information based on the sequence information obtained in step a) and the information stored by the method according to claim 1.
